(19) United States
(12) Patent Application Publication
(10) Pub. No.: US 2010/0285532 A1
(43) Pub. Date: Nov. 11, 2010

(54) PRODUCTION OF HETEROLOGOUS PROTEINS OR PEPTIDES

(76) Inventors: Eldie Berger, Lynnwood Manor (ZA); Erika Margarete Du Plessis, Pretoria (ZA); Maureen Elizabeth Louw, Pretoria (ZA); Michael Craig Crampton, Garsfontein (ZA); Isak Bartholomeus Gerber, Krugersdorp (ZA)

Correspondence Address: MCDERMOTT, WILL & EMERY, 11682 EL CAMINO REAL, SUITE 400, SAN DIEGO, CA, 92130-2047, US

(21) Appl. No.: 12/812,023

(22) PCT Filed: Dec. 8, 2008
(86) PCT No.: PCT/IB08/55143

§ 371 Date: Jul. 7, 2010

(30) Foreign Application Priority Data
Jan. 8, 2008 (ZA) 2008/00205

(51) Int. Cl. C12P 21/00; C07K 14/195; C07H 21/04; C12N 15/63; C12N 1/21

(52) U.S. Cl. 435/69.7; 530/350; 536/23.7; 435/320.1; 435/252.3; 435/252.31

(57) ABSTRACT

A method of producing a flagellin-based chimeric protein includes culturing a B. halodurans BhFD05 (Δhag, ΔfliD, ΔwprA, Δalp, Δapr, Δvpr, Δasp) strain deposited under Accession Number 41533 at the NCIMB. The strain is caused to express and secrete high levels of a flagellin-based chimeric protein into an extracellular growth medium. The flagellin-based chimeric protein comprises a heterologous peptide (i) inserted in-frame into a flagellin variable region which is flanked on its N-terminal side by an N-terminal fragment of a flagellin polypeptide and, optionally, flanked on its C-terminal side by a C-terminal fragment of a flagellin polypeptide, or (ii) fused to the C-terminal of a flagellin polypeptide.

THIS INVENTION relates to the production of heterologous proteins or peptides by Gram-positive bacterial host cells.

### BACKGROUND

PCT International application PCT/IB2005/054022 (International publication number WO 2006/072845) describes recombinant Gram-positive bacterial strain (B. halodurans Alk36) which has the ability to over-produce flagellin protein (FliC) when compared to other Gram-positive bacterial strains. The recombinant strain produces high levels of stable and soluble recombinant flagellin protein on the cell surface of the recombinant strain. In order to achieve this, the recombinant strain is genetically modified to facilitate the expression of a chimeric polypeptide comprising a flagellin monomer and a peptide of choice inserted into the central variable region thereof. The genetic modifications include (i) Inactivating the hag gene on the chromosome which codes for functional flagellin; (ii) inactivating the cell wall protease wprA; and (iii) transforming the recombinant strain with a multicopy vector containing the gene encoding an in-frame flagellin peptide fusion protein.

### SUMMARY OF INVENTION

The inventors have developed a method for making therapeutic peptides utilizing a modified flagella type III secretion system whereby the therapeutic peptides are exported into the growth medium by a modified B. halodurans Alk36 strain (NCIMB 41533).

The modifications include inactivation of the flagellin gene (hag gene) by a disruption preventing expression of a functional flagellin. The disruption can be by replacement of an endogenous gene with a DNA sequence encoding either no polypeptide or a non-functional flagellin polypeptide. In this case, the non-functional flagellin polypeptide is a deletion mutant lacking amino acids 14 to 226 of SEQ ID NO: 1. The disruption of the hag gene is fully described in the PCT International application PCT/IB2005/054022 (International publication number WO 2006/072845), which is fully herein incorporated by reference.

Export of chimeric flagellin monomers was achieved by altering the genome of the B. halodurans (Δhag) strain through targeted inactivation of a fliD gene encoding a flagellin cap protein in addition to the genetic modification disclosed in the PCT International application PCT/IB2005/054022 (International publication number WO 2006/072845). The cap protein aids polymerization of the flagellin monomers to form a flagellin filament. The cap protein comprises 5 FliD subunits located at the tip of the flagellin filament and needs to be in place for polymerization of flagellin protein to take place. Inactivation of the fliD gene results in secretion of un-polymerized chimeric flagellin monomers into the extracellular medium. In this case, the non-functional FliD polypeptide is of SEQ ID NO: 2.

Protease gene homologues to the key proteases as identified in B. subtilis from the literature were selected for gene targeted inactivation. The sequences were identified from a search of the B. halodurans C-125 genome as accessed from the DNA Data Bank of Japan (DDBJ; http://gib.genes.nig.ac.jp). These include wprA (BH2080), alp (BH0684), vpr (BH0831), apr (BH0696), asp (BH0855) and aprX (BH1930) genes.

In order to improve the secretion ability of strain B. halodurans Alk36, its genome was further altered through targeted inactivation of these key protease genes. The resultant strain B. halodurans Alk36 (Δhag, ΔfliD, ΔwprA, Δalp, Δapr, Δvpr, Δasp), designated BhFD05, was transformed with an expression vector containing a fusion polypeptide linked to either the N-terminal or C-terminal flagellin region(s) or situated in a flagellin variable region, linked to both the N-terminal and C-terminal regions.

Thus, in accordance with a first aspect of the invention, there is provided a method of producing a flagellin-based chimeric protein, the method including
culturing a B. halodurans BhFD05 strain deposited under Accession Number 41533 at the NCIMB, and
causing the strain to express and secrete a flagellin-based chimeric protein into an extracellular growth medium,
wherein the flagellin-based chimeric protein comprises a heterologous peptide (i) inserted in-frame into a flagellin variable region which is flanked on its N-terminal side by an N-terminal fragment of a flagellin polypeptide and, optionally, flanked on its C-terminal side by a C-terminal fragment of a flagellin polypeptide, or (ii) fused to the C-terminal of a flagellin polypeptide.

The N-terminal-, C-terminal- and variable regions of the B halodurans flagellin protein are as defined in PCT International application PCT/IB2005/054022 (International publication number WO 2006/072845).

B. halodurans BhFD05 was deposited under Accession Number NCIMB41533 on 17 Dec. 2007 at NCIMB Ltd of Furguson Building, Craibstore Estate, Buchsburn, Aberdeen AB210YA.

The growth medium containing the chimeric protein may be usable as a crude preparation. The crude preparation may be a cell-free preparation.

The method may include partially or fully purifying the chimeric protein from the growth medium.

According to a second aspect of the invention, there is provided a flagellin-based chimeric protein produced by the method of the first aspect of the invention, and which comprises a heterologous peptide (i) inserted in-frame into a flagellin variable region which is flanked on its N-terminal side by an N-terminal fragment of the flagellin polypeptide and, optionally, flanked on its C-terminal side by a C-terminal fragment of a flagellin polypeptide, or (ii) fused to the C-terminal of a flagellin polypeptide.

The heterologous peptide may be fused to only the N-terminal fragment of the flagellin polypeptide.

Instead, the heterologous peptide may be fused to the C-terminal of a full length flagellin polypeptide.

The flagellin-based chimeric protein may instead, or additionally, include a polypeptide tag fused to the N-terminal of the heterologous peptide. Such a tag may be used to isolate the chimeric protein. The tag may also be used as a specific target in Western blot analysis. The tag may be a known tag such as a FLAG-tag, a HIS-tag, or the like. More than one copy of the tag may also be fused to the N-terminal of the heterologous peptide.

The flagellin-based chimeric protein may instead, or additionally, include a cleavage site adjacent to at least one side of, or linked to, the heterologous region, ie the heterologous peptide. A cleavage site may be provided adjacent to both sides of the heterologous region, i.e. cleavage sites may flank the heterologous region. The cleavage site(s) may be known cleavage sites such as a methionine cleavage site which is recognised by chemical agents such as cyanogen bromide.

The heterologous peptide (or polypeptide) may be a therapeutic peptide, which may be selected from the group consisting of an antimicrobial peptide, an antiviral peptide and an immunogenic peptide.

When the heterologous peptide is an antimicrobial peptide, it may be a cationic peptide. The cationic peptide may be Indolicidin.

When the heterologous peptide is an antiretroviral peptide, it may be ‘Enfuvirtide’ which is marketed as “Fuzeon” (trademark). Instead, it may then be “Sifuvirtide” which is profiled as a promising improvement to Fuzeon.

When the heterologous peptide is an immunogenic peptide, it may be an HIV antigenic peptide. The HIV peptide may be a consensus sequence of the variable region of all HIV-1 subtype C V3 South African isolates.

The size of the heterologous peptides expressed ranged from 12- to 75 amino acids. Yields obtained after tag purification from the different constructs ranged from 2-20 mg/L.

The invention extends further to the use of the flagellin-based chimeric protein according to the second aspect of the invention, in the manufacture of a medicament for therapeutic use.

According to a third aspect of the invention, there is provided a nucleic acid encoding a chimeric protein according to the second aspect of the invention, the nucleic acid comprising a nucleotide sequence encoding (i) the N-terminal fragment of a flagellin polypeptide; the variable region of a flagellin polypeptide; optionally, the C-terminal fragment of a flagellin polypeptide, and a nucleotide sequence encoding a heterologous peptide inserted in-frame into the nucleotide sequence encoding the variable region of the flagellin polypeptide, or (ii) a heterologous polypeptide or therapeutic peptide fused to the C-terminal of a flagellin polypeptide.

The nucleic acid may include a nucleotide sequence encoding the N-terminal fragment of the flagellin polypeptide ligated on its C-terminal end in-frame to a nucleotide sequence encoding a heterologous peptide.

The nucleotide sequence encoding the heterologous peptide may be inserted immediately after any nucleotide between nucleotide 162 and nucleotide 606 of SEQ ID NO: 3.

The nucleotide sequence encoding the heterologous peptide may be inserted as an in-frame fusion immediately after nucleotide 816 of SEQ ID NO. 3.

According to a fourth aspect of the invention, there is provided an expression cassette which includes a nucleic acid sequence encoding the chimeric protein according to the second aspect of the invention.

The nucleic acid sequence encoding the chimeric protein may be integrated into the chromosome of the host cell.

According to a fifth aspect of the invention, there is provided a nucleic acid vector which includes a nucleic acid sequence encoding the chimeric protein of the second aspect of the invention, operably linked to a transcriptional regulatory element (TRE).

The nucleic acid vector may be an extra-chromosomal plasmid.

According to a sixth aspect of the invention, there is provided a bacterial cell containing the nucleic acid vector of the fifth aspect of the invention.

The bacterial cell may be a Gram-positive bacterial cell such as a cell of the Bacillus genus, e.g., a cell of the B. halodurans species. The cell may be of the strain B. halodurans BhFD05 (Δhag, ΔfliD, ΔwprA, Δalp, Δapr, Δvpr, Δasp) deposited under Accession Number 41533 at the NCIMB.

The terms “polypeptide” and “protein” are used interchangeably to mean any peptide-linked chain of amino acids, regardless of length or post-translational modification.

Unless otherwise defined, all technical and scientific terms used herein have the same meaning as commonly understood by one of ordinary skill persons in the art to which this invention pertains. In case of conflict, the present document, including definitions, control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Further features of the invention will now be described with reference to the following non-limiting examples, sequence listings and accompanying drawings.

In the drawings,

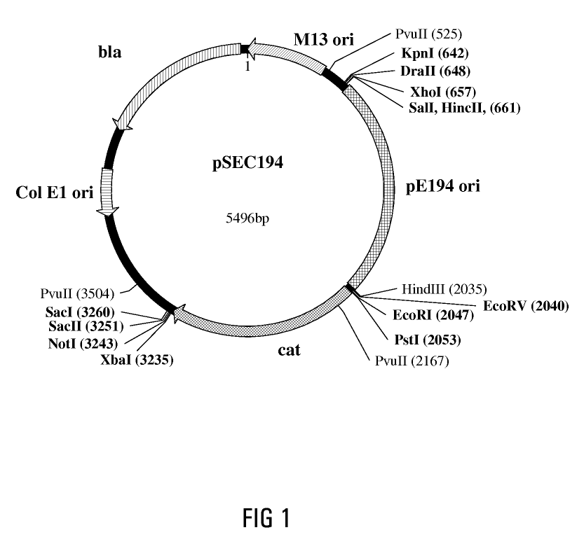

FIG. 1: Plasmid map of pSEC194 (5.496 kb). Temperature sensitive or (pE194) and ColE1 oh used for replication in Bacillus and E. coli respectively. Restriction enzyme sites (bold), useful for cloning. Plasmid map created with DNAMAN, Version 4.1;

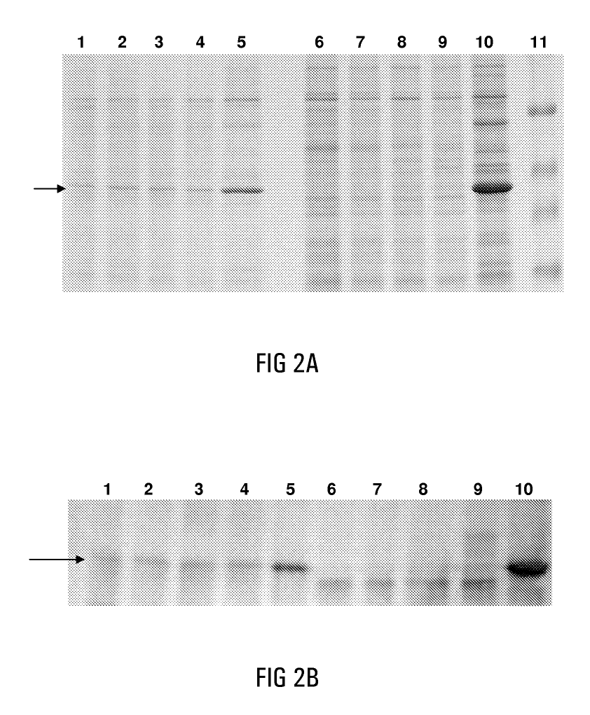

FIG. 2A: SDS-PAGE comparison of extracellular samples from different protease deficient B. halodurans strains secreting HIV antigenic fusion peptide at pH 8.5 during exponential (lanes 1-5, OD₆₀₀ 1.6) and stationary (lanes 6-10, OD₆₀₀ 5) phase. Lanes 1 and 6, strain BhFD01, lanes 2 and 7, strain BhFD02; lanes 3 and 8, BhFD03; lanes 4 and 9, BhFD04; lanes 5 and 10, BhFD05 and lane 11, molecular weight marker (Fermentas). The arrow indicates the HIV antigenic fusion peptide;

FIG. 2B. Western blot using anti-FLAG antibodies against the chimeric flagellin carrying the HIV antigenic peptide. The arrow indicates the FLAG HIV antigenic fusion peptide;

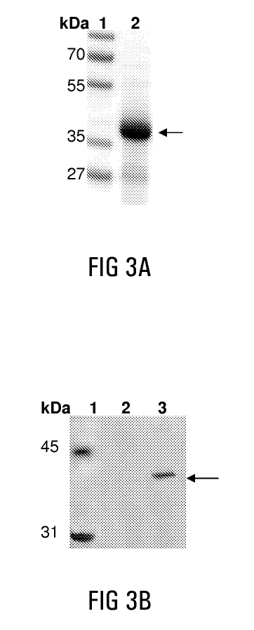

FIG. 3A: SDS PAGE gel analysis of the extracellular protein fraction of chimeric flagellin in B. halodurans strain BhFD05 after chromatography. Lane 1, molecular weight marker (Fermentas); lane 2, pSECNHIVC7 FLAG-affinity eluate. The arrow indicates the flagellin-HIV antigenic fusion protein;

FIG. 3B: Western blot analysis of 3A using MEIV3b 4 antibodies. Lane 1, low molecular mass marker (Biorad); lane 2, pSECNC7 (negative control) and lane 3, pSECNHIVC7. The arrow indicates the flagellin-HIV antigenic fusion protein;

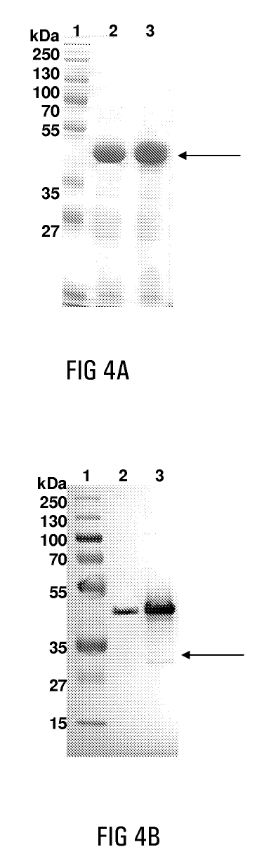

FIG. 4A: SDS PAGE gel analysis of the extra-cellular protein fraction of chimeric flagellin carrying the FLAG-tag in B. halodurans strain BhFD05 after FLAG-affinity chromatography. Lane 1, low molecular mass ladder (Fermentas); lane 2, pSECNFFuzC7 and lane 3, pSECNF2SifC7. The arrow indicates Fuzeon™ and Sifuvirtide flagellin fusion proteins;

FIG. 4B: Western blot analysis using anti-FLAG antibodies against the extracellular protein fractions from the chimeric flagellin expression cassettes in B. halodurans BhFD05. Lane 1, low molecular mass marker; lane 2, pSECNF2SifC7; and lane 3, pSECNFFuzC7. The arrow indicates Fuzeon™ and Sifuvirtide flagellin fusion proteins;

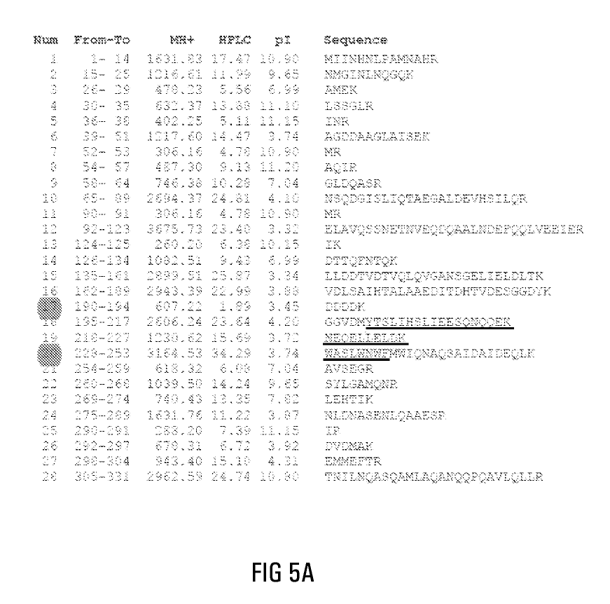

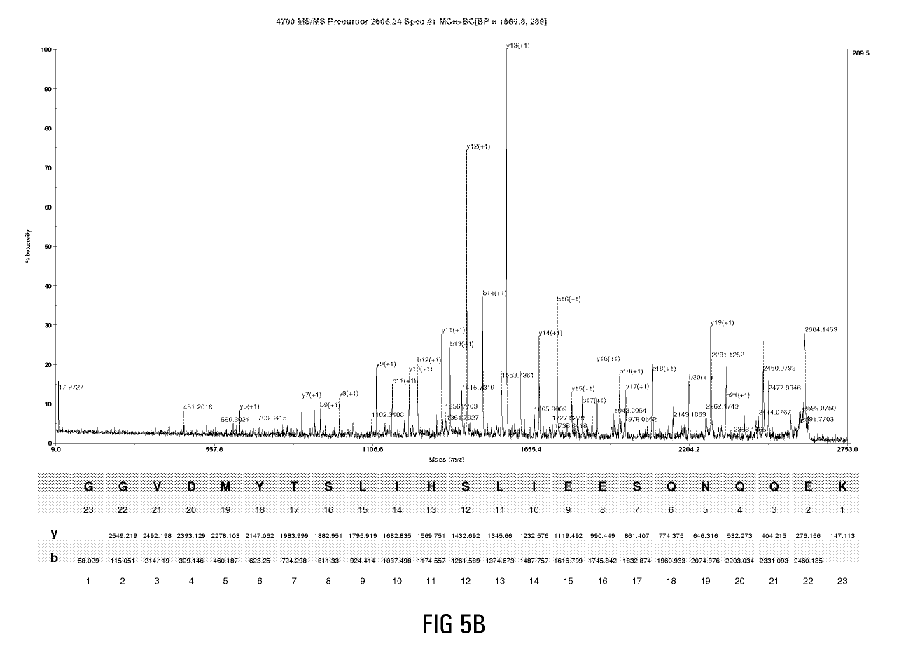

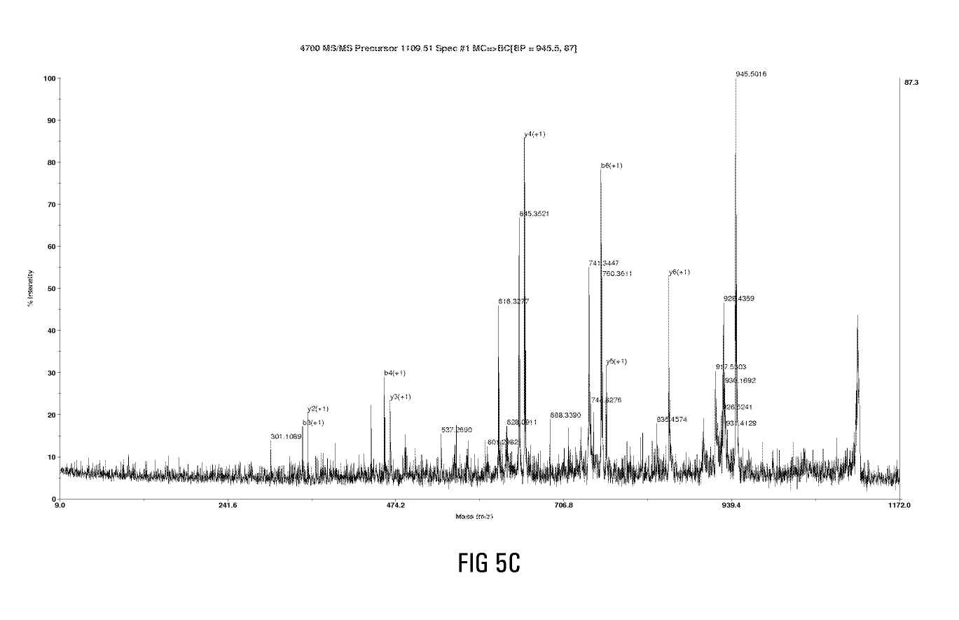

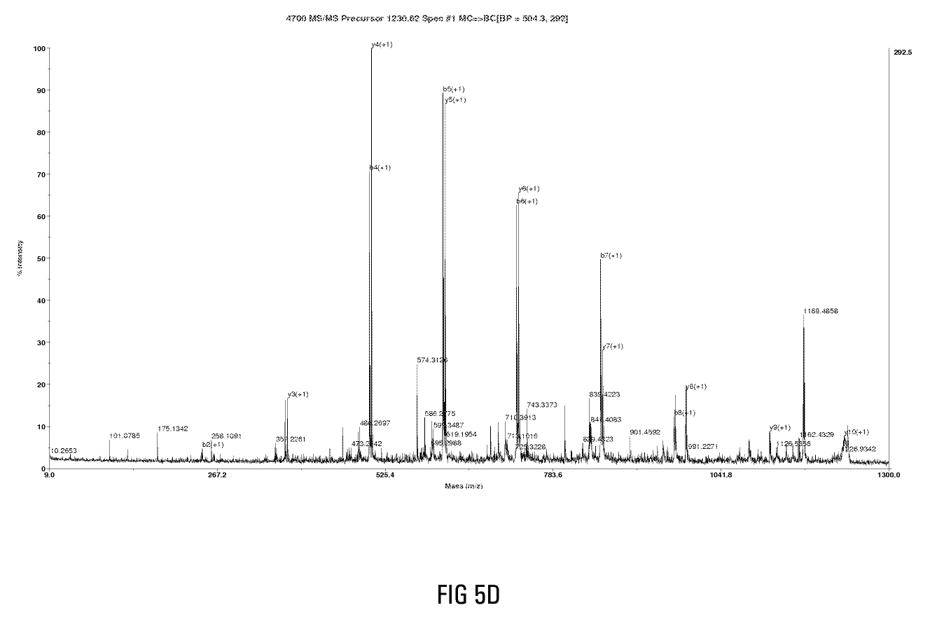

FIG. 5: Mass spectrometry (MS)-based data for the verification of Fuzeon expression and integrity. (A) Cleavage report and identified peptides of the chimeric flagellin gene product of pSECNFFuzC7. The sequence of the peptide of interest, Fuzeon™, is underlined. (B) MS/MS spectrum of the mass 2606.240 confirming the presence of GGVDMYTSLIHSLIEESQNQQEK, the N-terminal region of Fuzeon™ peptide. (C) MS/MS spectrum of the mass 1109.51 confirming the presence of WASLWNWF, the C-terminal end of Fuzeon peptide. (D) MS/MS spectrum of the mass at 1230.62 confirmed to be the fragment NEQELLELDK of the Fuzeon™ peptide;

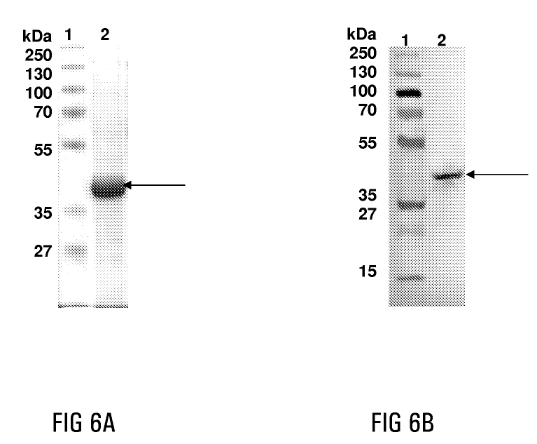

FIG. 6A: SDS PAGE gel analysis of the extra-cellular protein fraction of chimeric flagellin carrying the FLAG-tag in B. halodurans strain BhFD05 after FLAG-affinity chromatography. Lane 1, low molecular mass ladder; lane 2, pSECNFINDC7. The arrow indicates the Indolicidin flagellin fusion protein;

FIG. 6B: Western blot of the chimeric flagellin carrying the pSECNFINDC7 FLAG-tag. Lane 1, molecular weight marker (Fermentas); lane 2, pSECNFINDC7 FLAG-affinity eluate. The arrow indicates the Indolicidin flagellin fusion protein;

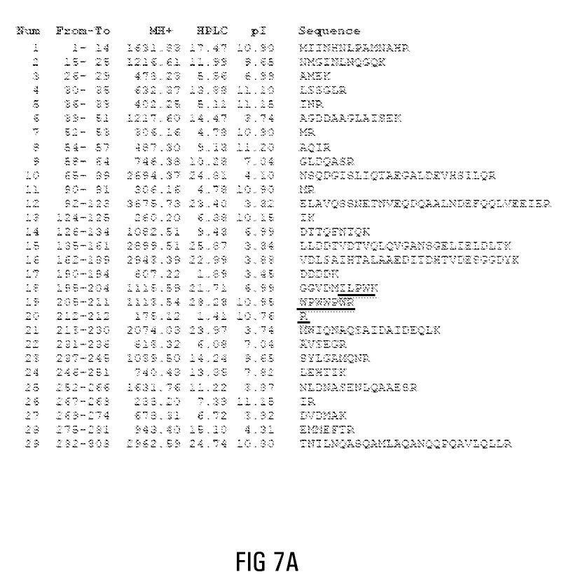

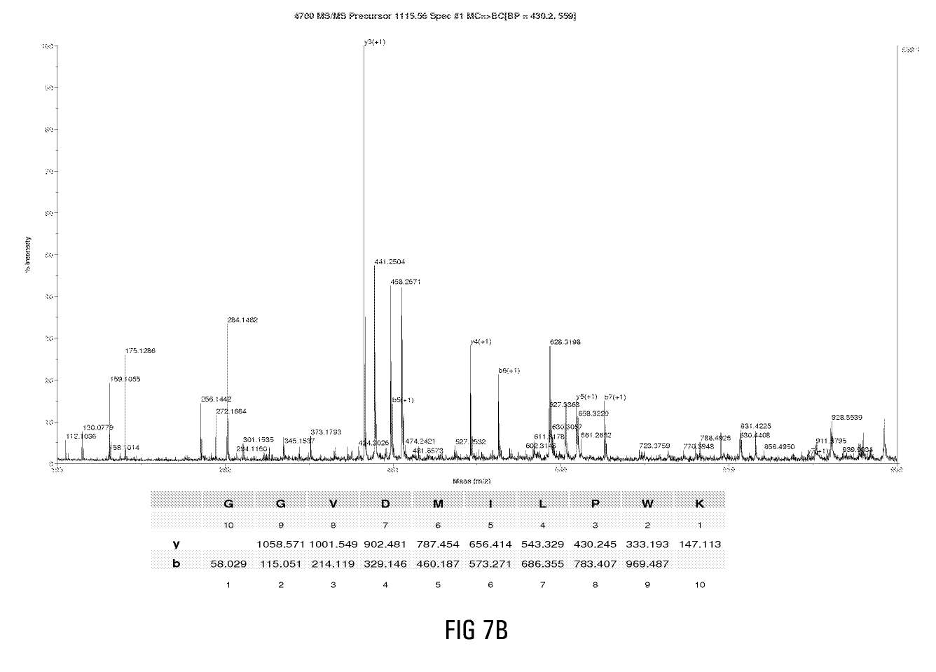

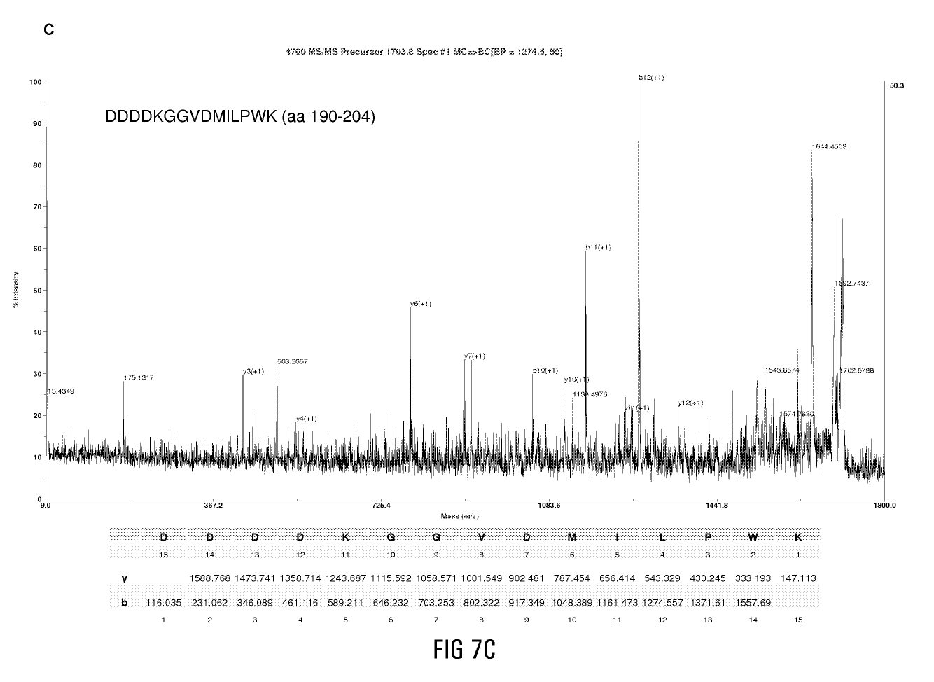

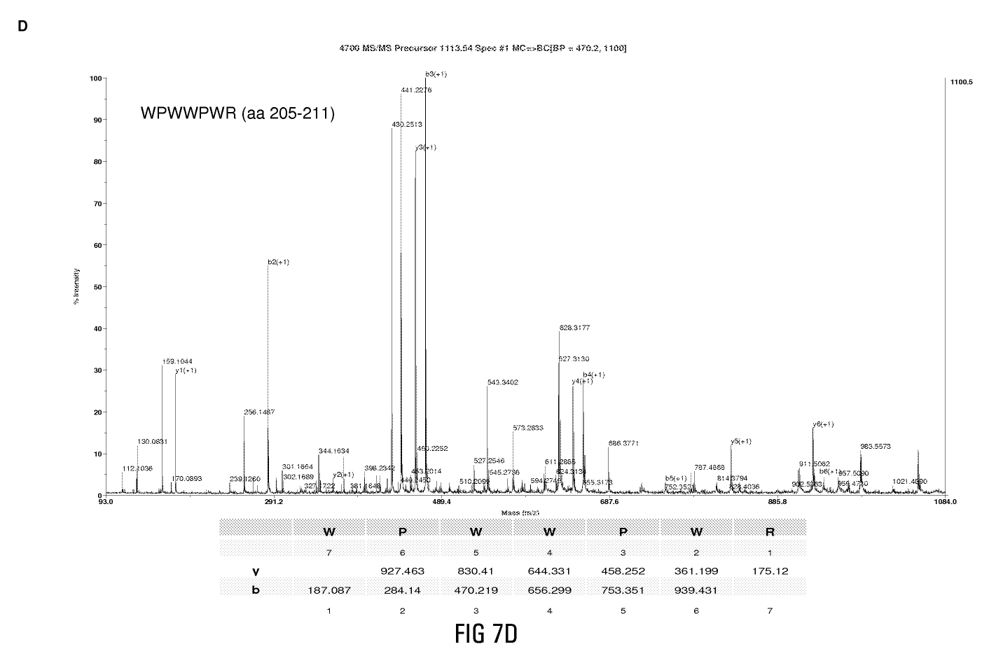

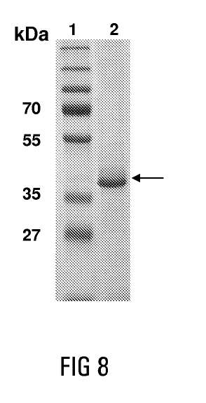

FIG. 7: Mass spectrometry (MS)-based data for the verification of Indolicidin expression and integrity. (A) Cleavage report and identified peptides of the chimeric flagellin gene product of pSECNFINDC7. The sequence of the peptide of interest, Indolicidin, is underlined. (B) MS/MS spectrum of the mass 1115.59 Da confirming the presence of (GGVDMILPWK, aa 195-204), the N-terminal region of Indolicidin peptide. (C) MS/MS spectrum of the mass 1703.79 Da relating to the N-terminal part of Indolicidin, DDDDKGGVDMILPWK (aa 190-204) generated by a missed cleavage after K194, encompassing both of the tryptic peptides not clearly evident from the cleavage report shown in FIG. 8A. (D) MS/MS spectrum of the mass at 1113.542 confirming the presence of the peptide WPWWPWR (aa 205-211) of the Indolicidin peptide;

FIG. 8: SDS-PAGE gel showing FLAG-tag purification of extra-cellular supernatant from strain BhFD05 (pSECNF2Sif) OD₆₀₀ 4.0. Lane 1, molecular weight marker (Fermentas); lane 2, FLAG-affinity eluate; The arrow indicates NF2Sif peptide;

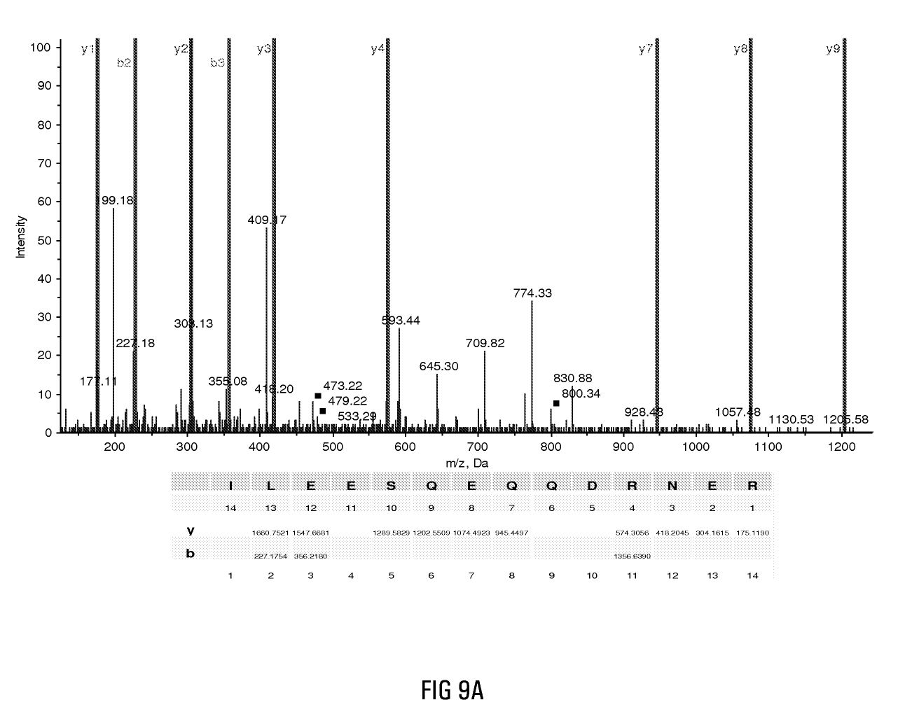

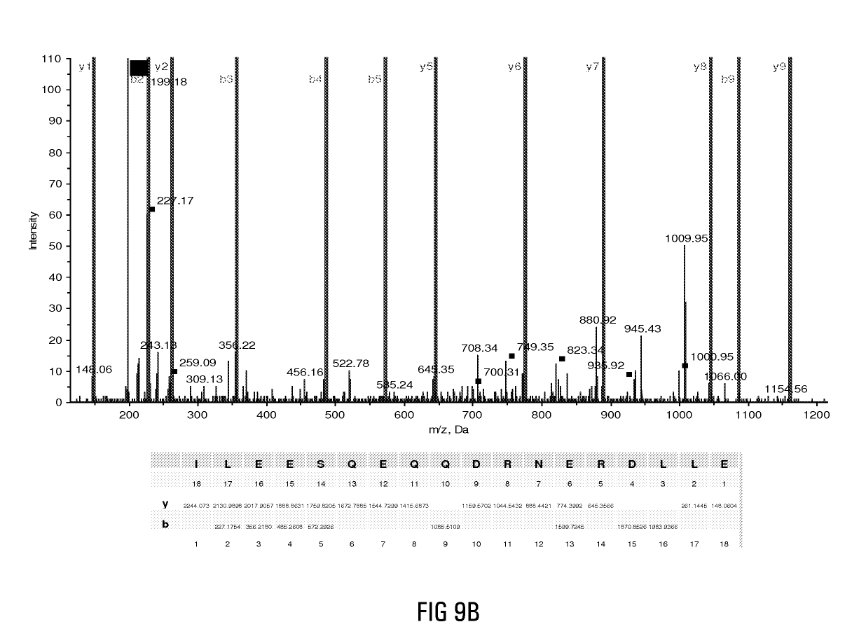

FIG. 9: Mass spectrometry (MS)-based data for the verification of the chimeric flagellin gene product of pSECNF2Sif, Sifuvirtide. MS/MS spectra are indicated to confirm the presence of the masses at 1772.8372 Da, ILEESQEQQDRNER (A) and 2243.0657 Da, ILEESQEQQDRNERDLLE (B) for overlapping peptide sequences making up the C-terminal end of Sifuvirtide;

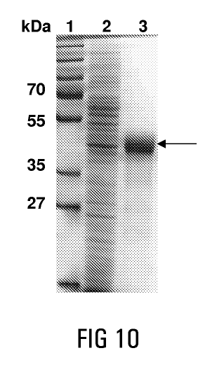

FIG. 10: SDS-PAGE gel showing FLAG-tag purification of extra-cellular supernatant from strain BhFD05 (pSECNCF2Sif) OD₆₀₀ 4.0. Lane 1, molecular weight marker (Fermentas); lane 2, Crude sample and lane 3, FLAG-affinity eluate; The arrow indicates NCF2Sif peptide; and

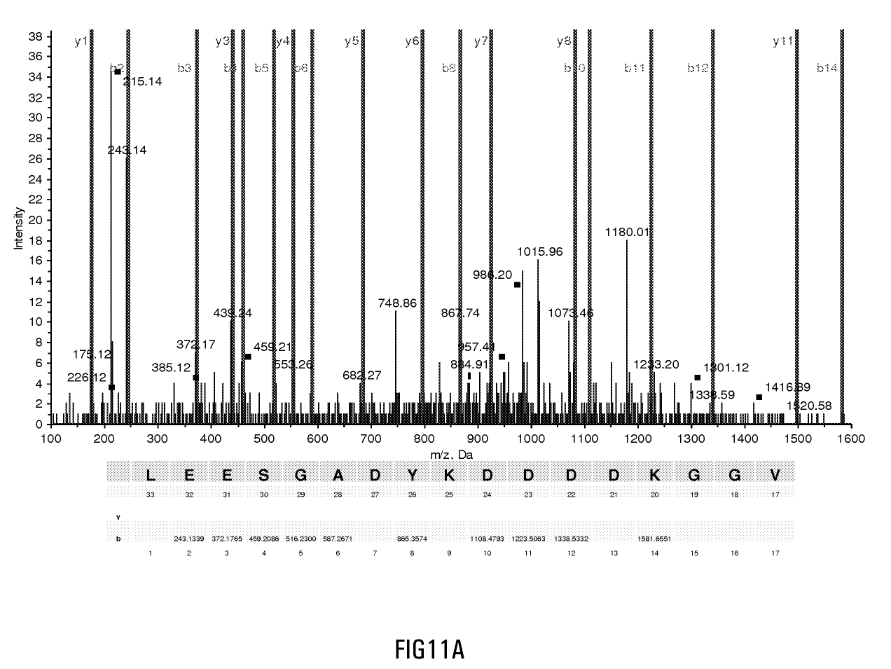

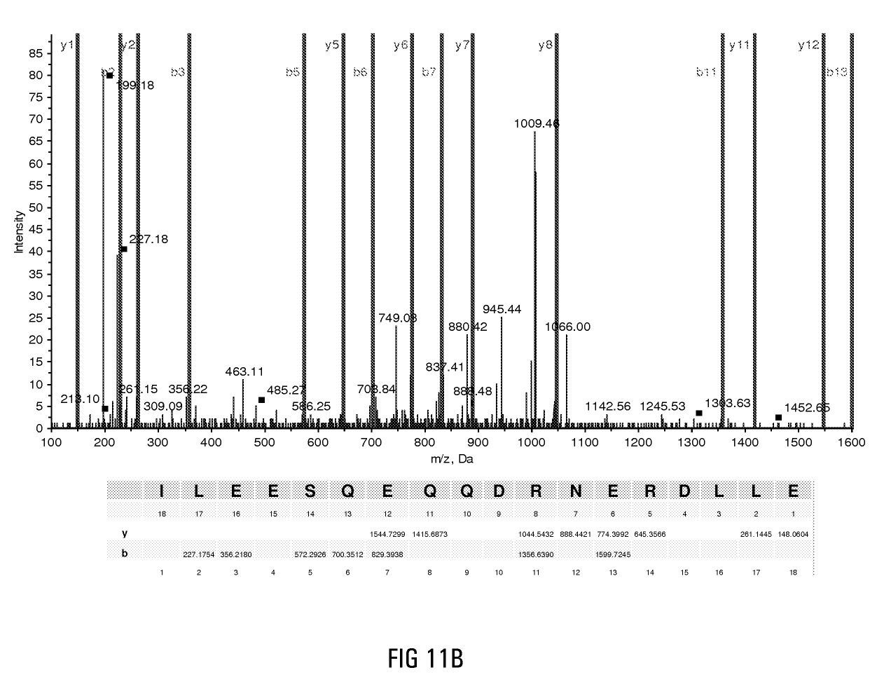

FIG. 11: Mass spectrometry (MS)-based data for the verification of the chimeric flagellin gene product of pSECNCF2Sif, Sifuvirtide. MS/MS spectra are indicated to confirm the presence of the masses at 3938.6768 Da, the N-terminal region of the anti-viral peptide, LEESGADYKDDDDKGGVDMSWETWEREIENYTR (A); as well as 2243.0669 Da, the C-terminal region, ILEESQEQQDRNERDLLE (B).

### DEVELOPMENT OF HOST GENETIC BACKGROUND

### Example 1

### Inactivation of the fliD gene on the chromosome of B. halodurans BhFC04 (Δhag ΔwprA)

Primers were designed to amplify two fragments of the fliD gene by PCR amplification. These were 1.5 kb and 0.989 kb respectively and contained part of the N-terminal (primers σ^{D}Kpn/MC120805, Table1) and part of the C-terminal (primers FliDCF2/FliDCR2, Table1) regions of the fliD gene. The vector pSEC194 (Crampton et al. 2007) was digested with KpnI/HindIII and ligated to both fragments in a 3 way ligation and transformed into E. coli DH10B to create the plasmid pSECFliD containing the defective fliD gene. This plasmid was then transformed into B. halodurans BhFC04 (Δhag, ΔwprA) and integration was according to Crampton et al (2007). Twenty putative single crossover colonies were screened with primers M13F and DChrRev (Table1). Five N-terminal single crossover clones were obtained and 15 C-terminal single crossover clones. One of the N-terminal crossover colonies was used to create a double crossover. PCR amplification with primers ChrFliFor and DChrRev (Table1) proved that the double crossover event did occur. Twenty chloramphenicol sensitive colonies were tested and 12 of them proved to be correct—containing the defective fliD gene while the rest were found to be revertants. This strain was named B. halodurans BhFD01 (Δhag, ΔwprA, ΔfliD).

### Example 2

### Inactivation of Key Protease Genes on the Chromosome

To further improve the stability of secreted chimeric peptide monomers by decreasing proteolytic degradation it was decided to inactivate proteases other than the wprA cell wall protease on the B. halodurans BhFD01 chromosome. The protease genes targeted were: alp, apr, asp and vpr

2.1 Inactivation of the Prepro-Alkaline Protease (alp) Gene of B. halodurans Alk36 (BhFD01).

The alp gene is located at position 740001 to 741119 on the B. halodurans C125 genome (http://www.jamstec.co.jp/genomebase/micrhome). The primers used to generate the two PCR products alp1 (alp1F/alp1R) and alp2 (alp2F/alp2R) needed for construction of the defective alp-fragment are listed in Table 1. The 1183 by alp1 PCR product started 999 bps upstream of the ATG start codon and included the first 184 bps of the alp N-terminal region.

The 848 by alp2 PCR product included the last 161 bps of the alp C-terminal region as well as 687 bps downstream of the TAA stop codon of the alp gene.

TABLE 1
List of PCR primers and their
corresponding nucleotide sequences. Restriction
enzyme sites are underlined.

| | | Restriction |
|---|---|---|
| Primer | | enzyme |
| name | Nucleotide sequence | sites |
| alp1F | 5CTTGGTACCGCGTGGGAATGTTGCA3′ | KpnI |
| alp1R | 5′CTTGGATCCTGCACTTCTACCGCTG | BamHI |
| | AG3′ | |
| alp2F | 5′CTTGGATCCGGCTTCACCTCATGT | BamHI |
| | GA3′ | |
| alp2R | 5′CTCCCGGGTGGTTGTCACAGCAGC | SmaI |
| | GG3′ | |
| apr1F | 5′GCAGGTACCGTTGGTGTTCAAGATG | KpnI |
| | TTTACG-3′ | |
| apr1R | 5′GCAGGATCCAGGCGTTGCTTGAGAC | BamHI |
| | GTACCA3′ | |
| apr2F | 5′GCAGGATCCGGACAGGAAGCGAACC | BamHI |
| | TCAAG3′ | |
| apr2R | 5′GCACCCGGGCAAGTCCTAGAGTACA | SmaI |
| | ATAAC3′ | |
| vpr1F | 5′CGTGTTACCGATGTGTAGTGCCTTA | KpnI |
| | TC3′ | |
| vpr1R | 5′CTTGGATCCTTCATACGTCTCGCCAT | BamHI |
| | CGAG3′ | |
| vpr2F | 5′CGTGGATCCCGAAGGTACGATCATC | BamHI |
| | GTA3′ | |
| vpr2R | 5′GTCCCGGGAAGCACGAGTGGATTCAT | SmaI |
| | GGTATA3′ | |
| asp1F | 5′GTAGGTACCCTCGATGCGAAAGTTCT | KpnI |
| | CGATG3′ | |
| asp1R | 5′GCAGGATCCGTACCAGCCACGTGAGT | BamHI |
| | TCCG3′ | |
| asp2F | 5′GCAGGATCCGCTAGATACTCTGGTGT | BamHI |
| | TATGG3′ | |
| asp2R | 5′GATCCCGGGCCTCCTATCATACCCAA | SmaI |
| | ATGAG3′ | |
| MC120805 | 5′CGAGGATCCCGTATTTAAAGAGGAAC | BamHI |
| | GTAA3′ | |
| FliDCF2 | 5′CGAGGATCCCGAGCAGTGATTACAGA | BamHI |
| | TTG3′ | |
| FliDCR2 | 5′CGACCCGGGCAGAGAGCTCATTATGC | SmaI |
| | TTCTC3′ | |
| DChrRev | 5′CTTAGATCATGGTTAGAATCAAGAG | — |
| | G3′ | |
| ChrFliFor | 5′GCTTGTGCTGGGCAAAGGAGGCGAA | — |
| | G3′ | |
| σ^{D}Kpn | 5′CTCGGTACCCTCGCGTTACGCTCTTT | KpnI |
| | CTGT3′ | |
| FliNterRev | 5′CTCCTCGAGCGACCTTCTGAAACA | XhoI |
| | GC3′ | |
| M13F | 5′TGACCGGCAGCAAAATG3′ | — |
| FliCR | 5′CAACAAAGTAACGGTTGAGCG3′ | — |
| FliDNR3 | 5′CGAGGATCCAAGACCGGCAGAGTTAA | BamHI |
| | TGTC3′ | |
| NC5F | 5′CACGTCGACTCGAGCCCGGGATCCTT | XhoI |
| | TAATACGCAAAAATTACTC3′ | |
| VCF6 | 5′CACGTCGACTCGAGCCCGGGATGGAT | SalI |
| | CCAGAATGCACAATCAGCTATTGAC3′ | |
| VNR6 | 5′GACGTCGACAGTGTGGTCAGTAATAT | SalI |
| | CCTC3′ | |
| FliDCR | 5′CGACCCGGGGAAGAAGCTGAAGACGA | SmaI |
| | TGCAGC3′ | |
| FliC7F | 5′CGAGGTACCAGGAGTTTGTCCTTC | KpnI |
| | TG3′ | |
| FuzendR | 5′CGTCAGGATCCTTACATAAACCAATT | BamHI |
| | CCA3′ | |
| FuzendR2 | 5′GTCTAGGATCC | |
| TermF | 5′GACGGATCCTTTGCTTCCATTTAA | BamHI |
| | AGATCT3′ | |
| TermR | 5′GTGCTGCAGGTATTTAAAGAGGAA | PstI |
| | CGTAAACG3′ | |
| SifRev | 5′CTCGAGGATCCTTATTCTAATAAA | BamHI |
| | TCACGTTC3′ | |

The digested PCR products (N-terminal KpnI/BamHI and C-terminal BamHI/SmaI) were ligated together into integration vector pSEC194 (KpnI and HindIII) to obtain pSECalp-harbouring the defective alp gene with the internal 771 bps deleted.

The defective alp-gene was integrated into the B. halodurans BhFD01 (Δ hag, ΔfliD Δ wprA) chromosome through a double crossover event as described in the previous section to create BhFD02 (Δhag, ΔfliD, ΔwprA, Δalp). The event was confirmed through PCR analysis.

2.2 Inactivation of the Prepro-Alkaline Protease (apr) Gene of B. halodurans.

The prepro-alkaline protease (apr) gene is located at position 751087 to 753465 on the B. halodurans C125 genome (http://www.jamstec.co.jp/genomebase/micrhome). The primers used to obtain the two PCR products needed for construction of the defective apr-fragment are (apr1F/apr1R) and (apr2F/apr2R) (Table 1). The 1625 by apr1 PCR product started 644 bps upstream of the TTG start codon of the apr gene and included the first 981 bps of the N-terminal sequence. The 1235 by apr2 PCR product included the last 167 bps of the apr gene C-terminal sequence as well as 1068 bps downstream of the TAA stop codon of the apr gene.

The apr PCR products (N-terminal KpnI/BamHI and C-terminal BamHI/SmaI) were ligated together into the vector pSEC194 (KpnI/HindIII) to obtain pSECapr-harbouring the defective apr gene with the internal 1231 bps deleted.

The defective apr gene was integrated into the B. halodurans BhFD02 (Δhag, ΔfliD, ΔwprA, Δalp) chromosome through a double crossover event as described previously to create BhFD03 (Δhag, ΔfliD, ΔwprA, Δalp, Δapr). This event was confirmed through PCR analysis.

2.3 Inactivation of the vpr Protease Gene of B. halodurans.

The vpr gene is located at position 905382 to 902983 on the B. halodurans C125 genome (http://www.jamstec.co.jp/genomebase/micrhome). The primers used to obtain the two PCR products needed for construction of the vpr-fragment are summarized in Table 1 (vpr1F/vpr1R and vpr2F/vpr2R). The 1724 by vpr1 PCR product started 578 bps upstream of the TTG start codon of the vpr gene and included the first 1146 bps of the vpr N-terminal sequence. The 1509 by vpr2 PCR product included the last 361 bps of the vpr gene C-terminal sequence as well as 1148 bps downstream of the TAA stop codon of the vpr gene. The temperature sensitive pSEC194 was restricted with KpnI and HincII and ligated to vpr1 and vpr2 to obtain pSECvpr.

The defective vpr gene was integrated into the B. halodurans BhFD03 (Δhag, ΔwprA, Δalp, Δapr) chromosome through a double crossover event as described previously. This event was confirmed through PCR analysis and created BhFD04 (Δhag, ΔfliD, ΔwprA, Δalp, Δapr, Δvpr).

2.4 Inactivation of the asp Protease Gene of B. halodurans.

The asp gene is located at position 927497 to 928582 on the B. halodurans C125 genome (http://www.jamstec.co.jp/genomebase/micrhome). The primers used to generate the two PCR products asp1 (asp1F/asp1R) and asp2 (asp2F/asp2R) needed for construction of the asp-fragment are listed in Table 1. The 852 by asp1 PCR product started 371 bps upstream of the ATG start codon and included the first 481 bps of the asp N-terminal region. The 682 by asp2 PCR product included the last 319 bps of the asp C-terminal region as well as 363 bps downstream of the TAA stop codon of the asp gene. The digested PCR products (N-terminal KpnI/BamHI and C-terminal BamHI/SmaI) were ligated together into integration vector pSEC194 (KpnI and HindIII) to obtain pSECasp—harbouring the defective asp gene with the internal 281 bps deleted. The defective asp gene was integrated into the B. halodurans BhFD04 (Δhag, ΔfliD, ΔwprA, Δalp, Δapr, Δvpr) chromosome through a double crossover event as described in the previous sections to obtain strain BhFD05 (Δhag, ΔfliD, ΔwprA, Δalp, Δapr, Δvpr, Δasp). This event was confirmed through PCR analysis.

### Application of Host Strain: Peptides Secreted According to the Invention as in-Frame Flagellin Sandwich Fusions

### Example 3

The effect of the fliD mutation on the secretion of chimeric flagellin fusions was evaluated with the following different peptides.
i) An HIV antigenic peptide (Table 2) of which the synthetic peptide sequence used was based on a consensus sequence of the variable region of all HIV-1 subtype C V3 South African isolates, Hewer and Meyer (2003). This peptide is 24 amino acids in size and the full insert size is 29 amino acids. The amino acid sequence of FliC including the HIV-antigenic peptide is provided as SEQ ID NO: 4. The construct which encodes the polypeptide sequence of SEQ ID NO: 4 was named pSECNHIVC7 and is provided as SEQ ID NO: 5.
ii) Indolicidin a cationic antimicrobial peptide (Table 2) of 13 amino acids (Hancock and Lehrer, 1998) was inserted as an in-frame chimeric flagellin fusion with a FLAG-tag inserted to facilitate isolation. The full insert size was 36 amino acids, which included methionine residues inserted either side of the Indolicidin peptide for cyanylation and selective cleavage commonly performed with cyanogen bromide (CnBr) (Tang and Speicher 2004). The amino acid sequence of FliC including the Indolicidin insert, with a FLAG-tag and cleavage sites, is provided as SEQ ID NO: 6. The construct which encodes the polypeptide sequence of SEQ ID NO: 6 was named pSECNFINDC7, and is provided as SEQ ID NO: 7.
iii) Enfuviritide is an HIV-fusion inhibitor that is marketed as Fuzeon™ and the synthetic oligonucleotides chosen were based on the amino acid sequence of enfuvirtide (Table 2) (Bolognesi et al. 1995). The peptide is 36 amino acids in size and a FLAG-tag was included to facilitate isolation as were methionine residues which were inserted either side of the peptide to facilitate cleavage. The total number of amino acids inserted into this construct was 59. The amino acid sequence of FliC including the enfuviritide insert, with a FLAG-tag and cleavage sites, is provided as SEQ ID NO: 8. The construct which encodes the polypeptide sequence of SEQ ID NO: 8 was named pSECNFFuzC7 and is provided as SEQ ID NO: 9.
iv) Sifuvirtide a 36 amino acid peptide (Table 2) is a promising alternative to enfuvirtide as it shows improved activity and stability (Franquelim et al. 2008). Methionine cleavage sites as well as two FLAG-tags were included giving a full insert size of 75 amino acids. The amino acid sequence of FliC including the Sifuviritide insert, with two FLAG-tags and cleavage sites, is provided as SEQ ID NO: 10. The construct which encodes the polypeptide sequence of SEQ ID NO: 10 was named pSECNF2SifC7 and is provided as SEQ ID NO: 11.

TABLE 2
HIV clade C, FLAG-Indolicidin, FLAG-Fuzeon and
FLAGx2-Sifuvirtide polypeptides inserted into
the NC7 site of the flagellin protein, the
FLAGx2-Sifuvirtide polypeptide ligated to the
N-terminal region of the flagellin protein and
the FLAG-Sifuvirtide polypeptide ligated to the
C-terminal of the flagellin protein

| Peptide Name | Peptide sequences |
|---|---|
| HIV antigenic | TRPNNNTRKSIRIGPGQTFYATGD |
| peptide | |
| FLAG-Indolicidin | DYKDDDDKGGVD [symbol] ILPWKWPWWPWRR [symbol] |
| (NFINDC7) | |
| FLAG-Fuzeon | DYKDDDDKGGVD [symbol] YTSLIHSLIEESQNQ |
| (NFFuzC7) | QEKNEQELLELDKWASLWNWF [symbol] |
| FLAGx2- | DYKDDDDKGGVDESGGDYKDDDDKGGVD [symbol] |
| Sifuvirtide | SWETWEREIENYTRQIYRILEESQEQQDRN |
| (NF2SifC7) | ERDLLE [symbol] |
| FLAGx2- | DYKDDDDKGGVDESGGDYKDDDDKGGVD [symbol] |
| Sifuvirtide | SWETWEREIENYTRQIYRILEESQEQQDRN |
| (NF2Sif) ligated | ERDLLE |
| to N-terminal | |
| FLAG-Sifuvirtide | DYKDDDDKGGVD [symbol] SWETWEREIENYTR |
| (NCFSif) ligated | QIYRILEESQEQQDRNERDLLE |
| to C-terminal | |

Bold amino acids indicate polypeptide and FLAG sequences, non-bold amino acids code for linkers and the shaded amino acids indicate the inserted methionine residues.

TABLE 3
List of complimentary oligonucleotide sequences
used in the construction of Indolicidin, Fuzeon ™,
and Sifuvirtide expression cassettes.

| Primer | | Restriction |
|---|---|---|
| Name | Oligonucleotide sequences | sites |
| HIVF | 5′ G GCT GTC GAC ACG CGT | |
| | CCA AAT AAT AAT ACG CGT | SalI |
| | AAA TCA ATT CGT ATT GGA | |
| | CCA GGA CAA ACG TTT TAT | |
| | GCA ACG GGA GAT TGG ATC CAG | |
| | GG 3′ | |
| HIVR | 5′ CC GCG GAT CCA ATC TCC CGT | |
| | TGC ATA AAA CGT TTG TCC TGG | BamHI |
| | TCC AAT ACG AAT TGA TTT ACG | |
| | CGT ATT ATT ATT TGG ACG CGT | |
| | GTC GAC GCC C 3′ | |
| IndF | 5′ CAG GTC GAC [symbol] ATC CTG | |
| | CCG TGG AAA TGG CCG TGG TGG | SalI |
| | CCG TGG CGT CGT [symbol] TGG | |
| | ATC CAG C 3′ | |
| IndR | 5′ G CTG GAT CCA [symbol] ACG ACG | |
| | CCA CGG CCA CCA CGG CCA TTT | SalI |
| | CCA CGG CAG GAT [symbol] GTC GAC | |
| | CTG 3′ | |
| FuzF | 5′-GCT GTC GAC [symbol] TAT ACG | SalI |
| | TCA TTA ATT CAT TCA TTA ATT | |
| | GAA GAA TCA CAA AAT CAA CAA | |
| | GAA AAA AAT GAA CAA GAA TTA | |
| | TTA GAA TTA GAT AAA TGG GCA | |
| | TCA TTA TGG AAT TGG TTT [symbol] | |
| | TGG ATC CAG G-3′ | |
| FuzR | 5′ C CTG GAT CCA [symbol] AAA | BamHI |
| | CCA ATT CCA TAA TGA TGC CCA | |
| | TTT ATC TAA TTC TAA TAA TTC | |
| | TTG TTC ATT TTT TTC TTG TTG | |
| | ATT TTG TGA TTC TTC AAT TAA | |
| | AGT ATG AAT TAA TGA CGT ATA | |
| | [symbol] GTC GAC AGC 3′ | |
| SifF | 5′ GCA GCT GTC GAC [symbol] | SalI |
| | TCA TGG GAA ACG TGG GAA CGT | |
| | GAA ATC GAA AAT TAT ACG CGT | |
| | CAA ATC TAT CGT ATC TTA GAA | |
| | GAA TCA CAA GAA CAA CAA GAT | |
| | CGT AAT GAA CGT GAT TTA TTA | |
| | GAA [symbol] TGG ATC CAG GTC 3′ | |
| SifR | 5′ GAC CTG GAT CCA [symbol] TTC | BamHI |
| | TAA TAA ATC ACG TTC ATT ACG | |
| | ATC TTG TTG TTC TTG TGA TTC | |
| | TTC TAA GAT ACG ATA GAT TTG | |
| | ACG CGT ATA ATT TTC GAT TTC | |
| | ACG TTC CCA CGT TTC CCA TGA | |
| | [symbol] GTC GAC AGC TGC 3′ | |
| FLAG F | 5′ TC GAC GAA TCT GGA GGA | SalI |
| | GAT TAT AAA GAT GAT GAT GAT | |
| | AAA GGA GGA G 3′ | |
| FLAGR | 5′ TC GAC TCC TCC TTT ATC | SalI |
| | ATC ATC ATC TTT ATA ATC TCC | |
| | TTC AGA TTC G 3′ | |

Bold nucleotides indicate polypeptides and FLAG sequences, non bold nucleotides code for the polylinker sequences and the shaded nucleotides indicate the inserted methionine residues. The underlined nucleotides correspond to the restriction enzyme sites.

These peptides were all inserted in the central variable region of the flagellin peptide monomer within the polylinker which was inserted at nucleoside position 540 in the flagellin gene (Crampton et al. 2007).

3.1 Construction of pSECNC7 Cassette.

This construct was an enhancement of construct pSECNC6 (described in PCT International Application PCT/IB2005/054022; International publication no. WO 2006/072845). The cassette pSECNC7 was obtained by PCR amplification of the N-(1447 bp) and C-terminal (1401 bp) regions of the flagellin gene with primers FliC7F/VNR6 and FliDNR3/VCF6 respectively (Table 1). The N-terminal and C-terminal products were digested with KpnI/SalI and SalI respectively. Both fragments were ligated into pSEC194 (KpnI/HincII) in a three way ligation to obtain pSECNC7 which contains the 27 base pair polylinker at nucleotide position 540 (FIG. 1).

### 3.2 Evaluation of the Expression of Immunogenic Peptides.

3.2.1 Construction and Evaluation of Immunogenic Chimeric Flagellin Fusion pSECNHIVC7 Secreted from B. halodurans BhFD05.

The plasmid pSECNHIVC7 (Table 2 and Table 3) containing the antigenic HIV peptide, construct as described by Crampton et al. (2007) was used and transformed into all five protease deficient strains; B. halodurans BhFD01, BhFD02, BhFD03, BhFD04 and BhFD05 and evaluated for secretion of the fusion peptide into the extracellular medium.

The chimeric flagellin gene product of pSECNHIVC7 in the different host backgrounds were evaluated for secretion into the extracellular medium. Cells were grown to an OD₆₀₀ of between 3.5 and 5.5 before harvesting. Approximately 1.5 ml of cell culture was harvested at 8000×g for 1 minute (supernatant contained extracellular fraction). 1 ml of the supernatant was removed and placed in an Eppendorf tube. 330 μl of a 20% TCA (trichloroacteic acid) solution was added to the supernatant and incubated on ice with shaking for 30 minutes. Extracellular proteins were pelleted at 12000×g for 10 minutes. The pellet was washed once with an ethanol and ether solution (1::1 v/v). Pellets were dried and proteins were resuspended in 30 μl 25 mM Tris-HCl solution (pH 9.5).

The extracellular protein fractions (10 μl) were analysed on a 10% SDS-PAGE gels and visualized using colloidal Coomassie stain (FIG. 2A). Approximately 1-2 μg was used for Western blot analysis (FIG. 2B).

3.2.2 Analysis of Immunogenic Chimeric Flagellin Fusions Secreted from B. halodurans BhFD05.

Cells were grown to an OD₆₀₀ of between 3.5 and 5.5 at 1 litre scale before harvesting the supernatant by centrifugation at 8000×g for 10 minutes. Total extracellular protein was precipitated from the supernatant by the addition of 10% (w/v) TCA (trichloroacteic acid) and incubation on ice for 30 minutes. Extracellular proteins were pelleted at 12000×g for 10 minutes. The pellet was washed once with an ethanol and ether solution (1:1 v/v). Pellets were air dried and proteins were resuspended in 10 ml 50 mM Tris-HCl solution (pH 7.4). The immunogenic chimeric flagellin fusion protein was purified to near homogeneity from the crude total extracellular protein fraction by anion exchange chromatography. The entire 10 ml crude protein preparation was loaded onto Toyopearl 650M strong anion exchange resin (Tosoh Bioscience) in a 1.6×13 cm pre-packed column using an ÄKTA FPLC™ protein purification system. Contaminating non-protein components were washed off the resin by 10-15 column volumes of wash buffer (50 mM Tris-HCl pH 7.4) until the baseline absorbance (A₂₈₀ₙₘ) stabilized around zero. Proteins were eluted off the resin by an increasing NaCl gradient ranging from 0-500 mM NaCl in 50 mM Tris-HCl, pH 7.4 over 15 column volumes. Protein-containing elution fractions were analysed on 10% SDS-PAGE gels to assess the final purity of the immunogenic chimeric flagellin fusion protein.

The purified immunogenic chimeric flagellin fusion protein was quantified using a Qubit™ fluorometer (Invitrogen) with Quant-iT™ protein assay reagents containing a highly sensitive protein-specific fluorescent dye and certified protein standards, used according to the manufacturer's instructions. The total protein quantity obtained was 9.87 mg immunogenic chimeric flagellin fusion protein per litre culture supernatant, which relates to ˜9.38 mg/L at an estimated 95% purity. After purification the chimeric flagellin fusion protein was analyzed on 10% SDS-PAGE gels as described above (FIG. 3A). Approximately 1-2 μg was used for Western blot analysis using MEIV3b 4 antibodies (FIG. 3B).

### 3.3 Evaluation of the Expression of Anti-Viral Peptides Fuzeon™ and Sifuvirtide.

3.3.1 Construction of pSECNFFuzC7 Carrying the FLAG::Fuzeon Peptide.

The synthetic oligonucleotides (Table 3) were designed based on the enfuvirtide (Fuzeon™) amino acid sequence (Table 2) (Bolognesi et al. 1995). A methionine residue was included at the N- and C-terminal end for chemical cleavage using cyanogen bromide (Tang and Speicher. 2004). A FLAG-tag was incorporated for ease of purification.

Oligonucleotides (Table 1) FuzF and FuzR were annealed according to the method described by IDT (Integrated DNA Technologies, www.idtdna.com). The oligonucleotides were diluted in STE buffer (10 mM Tris pH 8, 50 mM NaCl, 1 mM EDTA) to a final concentration of 20 μM. A working stock (5 μM) was made and equal amounts of complimentary oligonucleotides were mixed together (usually 25 μl of each). Samples were boiled for 5 minutes and allowed to cool very slowly in the waterbath. The resulting product was restricted with the appropriate restriction enzymes and ligated into pSECNC7 digested with SalI and BamHI. The resulting construct was named pSECNFuzC7. This construct was digested with SalI. FLAG-tag oligonucleotides were derived from the peptide sequence DYKDDDDK (Table 2) (Sigma cat no F3290)) with addition of a 4 and 2 amino acid linker at the N- and C-terminal ends for better FLAG-tag exposure. FLAG-tag oligonucleotides (Table 3) were annealed as described above. The annealed oligonucleotides were ligated to pSECNFuzC7 digested with SalI to obtain pSECNFFuzC7.

All constructs were confirmed to be correct by PCR analysis using primers FliNterRev and NC5F (Table 1). Only after confirmation of construct integrity were they transformed into B. halodurans BhFD05 using the modified protoplast transformation method (Crampton et al. 2007). Orientation of the insert was confirmed with restriction digests and directional PCR using primers FliCR and FuzF (Table 1 and Table 3).

3.3.2 Construction of pSECNF2SifC7 carrying a FLAG×2::Sifuvirtide Peptide.

The synthetic oligonucleotides were derived from the Sifuvirtide peptide sequence (Table 2) (Franquelim et al. 2008) and included a methionine residue at the N- and C-terminal ends for cyanogen bromide chemical cleavage. Oligonucleotides SifF and SifR (Table 3) were annealed according to the method described in section 3.3.1. The resulting product was restricted with the appropriate restriction enzymes (Table 1) and ligated into pSECNC7 digested with SalI and BamHI. The resulting construct was named pSECNSifC7.

pSECNSifC7 was digested with SalI. FLAG-tag oligonucleotides were annealed as described in section 3.3.1. The annealed oligonucleotides were ligated to pSECNSifC7 and resulted in the incorporation of two FLAG-tags in front of the Sif peptide. This construct was named pSECNF2SifC7.

All constructs were confirmed to be correct by PCR and sequencing analysis as described for pSECNFFuzC7. Orientation of the insert was confirmed by directional PCR using primers FliCR and SifF (Table 1 and Table 3). Constructs were then transformed into B. halodurans BhFD05.

3.3.3 Evaluation of Anti-Viral Chimeric Flagellin Fusions Secreted from B. halodurans BhFD05.

The chimeric flagellin gene products of pSECNFFuzC7 and pSECNF2SifC7 were evaluated for secretion into the extracellular medium. Cells were grown to an OD₆₀₀ of between 3.5 and 5.5 before harvesting. Approximately 1.5 ml of cell culture was harvested at 8000×g for 1 minute (supernatant contained extracellular fraction). 1 ml of the supernatant was removed and placed in an Eppendorf tube. 330 μl of a 20% TCA (trichloroacteic acid) solution was added to the supernatant and incubated on ice for 30 minutes. Extracellular proteins were pelleted at 12000×g for 10 minutes. The pellet was washed once with an ethanol and ether solution (1:1 v/v). Pellets were dried and proteins were resuspended in 30 μl 25 mM Tris-HCl solution (pH 9.5).

The extracellular protein fractions (10 μl) were analysed on a 10% SDS-PAGE gels and visualized using colloidal Coomassie stain. There were detectable chimeric flagellin protein bands for the flagellin fusions at the size range of 45 kDa (results not shown).

3.3.4 Analysis of Anti-Viral Chimeric Flagellin Fusions Secreted from B. halodurans BhFD05.

The desired flagellin-fusion protein was purified using affinity chromatography. The term “affinity chromatography” refers to a technique for separating molecules by their affinity to bind ligands attached to an insoluble matrix, so that the bound molecules can subsequently be eluted in a relatively pure state. The technique involved attaching a FLAG-tag to the fusion peptide, performing the chromatographic separation and isolating the fusion protein by excision from a (10%) SDS-PAGE gel.

The chimeric flagellin gene products of pSECNFFuzC7 and pSECNF2SifC7 were evaluated for secretion into the extracellular medium. Cells were grown to an OD₆₀₀ of between 3.5 and 5.5 at 1 litre scale before harvesting the supernatant by centrifugation at 8000×g for 10 minutes. Total extracellular protein was precipitated from the supernatant by the addition of 10% (w/v) TCA (trichloroacteic acid) and incubation on ice for 30 minutes. Extracellular proteins were pelleted at 12000×g for 10 minutes. The pellet was washed once with an ethanol and ether solution (1:1 v/v). Pellets were air dried and proteins were resuspended in 10 ml 50 mM Tris-HCl solution (pH 7.4) containing 500 mM NaCl and 2.5% (v/v) Triton X-100. The crude protein solution was incubated with 1 ml M2 Anti-FLAG affinity resin (Sigma) at room temperature for 2 hours to bind the FLAG-tagged fusion protein of interest. Non-specifically bound proteins were removed by washing the resin with 15 ml 50 mM Tris-HCl solution (pH 7.4) containing 500 mM NaCl and 2.5% (v/v) Triton X-100. The FLAG-tagged fusion protein was eluted with 8 ml 0.1M Glycine, pH 3.5 and analyzed on 10% SDS-PAGE gels as described in section 3.2.3 above (FIG. 4A). Approximately 1-2 μg was used for Western blot analysis using polyclonal rabbit anti-flagellin antibodies (FIG. 4B). The purified immunogenic chimeric flagellin fusion protein was quantified using a Qubit™ fluorometer (Invitrogen) with Quant-iT™ protein assay reagents containing a highly sensitive protein-specific fluorescent dye and certified protein standards, used according to the manufacturer's instructions. The total protein quantity obtained ranged between 10 and 20 mg immunogenic chimeric flagellin fusion protein per litre of culture supernatant, at an estimated 95% purity.

The anti-viral chimeric flagellin::Fuzeon protein band obtained from the pSECNFFuzC7 sample was excised from SDS-PAGE gels and subjected to enzymatic cleavage by modified sequencing grade trypsin through a method well known by those of skill in the art (Shevchenco and Shevchenco. 2001). This cleavage procedure cleaves polypeptides at the C-terminal side of lysine (K) and arginine (R) residues. The Fuzeon™ peptide presence and integrity were verified by mass-spectrometry analysis on an Applied Biosystems/MDS SCIEX 4800 MALDI TOF/TOF analyzer with CHCA (alpha-cyano-4-hydroxycinnamic acid) as the matrix and 1 fmol bradykinin as an internal calibrant (protonated, monoisotopic mass of 1060.5692). A preliminary examination of the mass spectra taken in reflector positive mode over the mass range 600-4000 Da, indicates that many of the highest peaks in each spectrum correspond to the tryptic peptides expected for the construct. A review of the cleavage report confirms that all expected masses within the range used in this mode (600-4000 Da) are present except for peptides 17 and 20 (FIG. 5A). Peptide mass matching was performed using GPMAW 7.1 with a precision of 50 ppm to check the coverage of the sequence in more detail. Twenty five masses were matched to 26 unmodified peptides, and 8 masses to 8 modified peptides, with 85% of the residues covered (284 of 331 amino acids). Each of these three precursors provided fairly complete y- and b-ion series. In order to further verify the sequence, several MS/MS spectra were acquired. Annotated MS/MS spectra are indicated for the peptides of interest with mass labels, y-ions and b-ions identified (FIGS. 5B, C, D). The peptide of interest spans the tryptic peptides expected at 2606.24, 1230.62, and 3164.53 Da (FIG. 5A). The peak at 2606.240 was identified as the peptide GGVDMYTSLIHSLIEESQNQQEK (aa 195-217, FIG. 5B) corresponding to the N-terminal region of Fuzeon™. No peak was evident at 3164.53 Da, but there was a peak at 1109.52 corresponding to the C-terminal portion of the inserted sequence. One peak in the MS spectrum stood out as a mass at 2074.03. An MS/MS spectrum of this mass was easily assigned to the peptide MWIQNAQSAIDAIDEQLK. This peptide does indeed occur in the fusion sequence, just after the inserted peptide of interest, and accounts for most of the peptide not covered by mass matching. The other half of the expected peptide should occur at 3164.53Da−2074.03Da+18Da+1Da=1109.5Da, where a peak of significant intensity was indeed present. This peak was confirmed by MS/MS to correspond to WASLWNWF (FIG. 5C), the C-terminal end of the Fuzeon™ peptide of interest. Furthermore, the mass at 1230.62 was confirmed to be NEQELLELDK (FIG. 5D) of the peptide of interest. In summary, there is positive proof that the peptide of interest, Fuzeon, (underlined, FIG. 5A) is indeed being expressed in its entirety as a chimeric flagellin fusion.

### 3.4 Evaluation of the Expression of Antimicrobial Peptide Indolicidin.

3.4.1 Construction of pSECNFINDC7 Carrying the FLAG::Indolicidin Peptide.

Synthetic Indolicidin oligonucleotides were derived from Selsted et al. (1992) and included a methionine residue at the N- and C-terminal ends for chemical cleavage (Table 2). Oligonucleotides (Table 3) were annealed as described in section 3.3.1. The resulting product was restricted with the appropriate restriction enzymes and ligated into pSECNC7 digested with SalI and BamHI resulting in pSECNINDC7. This construct was digested with SalI. FLAG-tag oligonucleotides were annealed as described in section 3.3.1. The annealed oligonucleotides were ligated to pSECNINDC7 and resulted in the incorporation of one FLAG-tag in front of the IND peptide. This construct was named pSECNFINDC7. The constructs were confirmed to be correct by PCR and sequencing analysis and transformed into B. halodurans BhFD05.

3.4.2 Analysis of Antimicrobial Chimeric Flagellin Fusions Secreted from B. halodurans BhFD05.

The antimicrobial peptide, Indolicidin, was extracted and purified from B. halodurans culture supernatant as described in section 3.3.4. The FLAG-tagged fusion protein of interest was isolated by affinity chromatography. The purified immunogenic chimeric flagellin fusion protein was quantified using a Qubit™ fluorometer (Invitrogen) with Quant-iT™ protein assay reagents containing a highly sensitive protein-specific fluorescent dye and certified protein standards, used according to the manufacturer's instructions. The total protein quantity obtained was between 1 and 5 mg immunogenic chimeric flagellin fusion protein per litre of culture supernatant, at an estimated 95% purity. Samples were run on a SDS-PAGE (10%) gel (FIG. 6A) and Western blot analysis using polyclonal rabbit anti-flagellin antibodies confirmed the results (FIG. 6B). The anti-microbial chimeric flagellin::Indolicidin protein band was excised from SDS-PAGE (10%) gels and subjected to cleavage by modified sequencing grade trypsin and MALDI TOF/TOF mass spectrometry analysis as described in section 3.3.4 above. A review of the cleavage report confirms that all expected masses within the range used in reflector positive MS mode (600-4000 Da) are present, except for peptides 17 and 18 (FIG. 7A). Peptide mass matching was performed using GPMAW 7.1 with a precision of 50 ppm to check the sequence coverage in more detail. 42 masses were matched to 39 unmodified peptides, and 17 masses to 16 modified peptides, with 99% of the residues covered (306 of 308 amino acids). In order to verify that the peptides of interest are being expressed in the fusion constructs, MS/MS spectra were obtained for several masses in the regions of interest. Annotated MS/MS spectra are indicated for the peptides of interest with mass labels, y-ions and b-ions identified (FIGS. 7B, C, D). The peptide expected at 1115.59 Da (GGVDMILPWK, amino acids 195-204) was identified and includes the N-terminal portion of the peptide of interest (FIG. 7B). To further demonstrate the presence of the N-terminal portion of the inserted sequence, an MS/MS spectrum was acquired of the precursor mass at 1703.79 Da (FIG. 7C). The identified peaks provide evidence for the peptide DDDDKGGVDMILPWK (amino acids 190-204) generated by a missed cleavage after K194, and encompassed both of the tryptic peptides not clearly evident from the cleavage report shown in FIG. 7A. The MS/MS spectrum of the precursor mass of 1113.542 confirming the presence of the peptide WPWWPWR (amino acids 205-211) is indicated in FIG. 7D. This peptide makes up the C-terminal region of Indolicidin. In summary, there is positive proof that the peptide of interest, Indolicidin, is indeed expressed in it's entirety as a chimeric flagellin fusion.

### Application of Host Strain: Peptides Secreted According to the Invention as N-Terminal Flagellin Fusions

### Example 4

### 4.1 Expression as FliC N-Terminal Fusion of the Anti-Viral Peptide, Sifuvirtide.

4.1.1 Construction of pSECNF2Sif Carrying the FLAG::Sifuvirtide Peptide.

The plasmid pSECNF2SifC7 was used as template for the primers σ^{D}Kpn and SifRev2 to obtain a 947 by PCR product containing the FliC N-terminal fragment (770 bp) fused to the 2×FLAG::Sifuvirtide peptide (225 bp) without a methionine residue at the C-terminal side of the Sifuvirtide peptide and the inclusion of a stop codon. The encoded polypeptide sequence of the FliC N-terminal fragment including the Sifuvirtide insert as described is provided as SEQ ID NO: 12. The same plasmid was used as a template with primers TermF and TermR to obtain a PCR product containing the FliC 3′ untranslated region (700 bp). These two fragments were digested with appropriate enzymes and ligated to pSEC194 digested with KpnI and HindII. The resulting construct, which encodes for the polypeptide sequence of SEQ ID NO: 12, was named pSECNF2Sif and is provided as SEQ ID NO: 13. The constructs were confirmed to be correct by PCR and sequencing analysis as described for pSECNFFuzC7 and then transformed into B. halodurans BhFD05.

### 4.1.2 Evaluation of the Expression of the N-Terminal Peptide Fusion.

The chimeric flagellin gene product of pSECNF2Sif was evaluated for secretion into the extracellular medium of B. halodurans BhFD05 according to section 3.3.3.

The extracellular protein fraction was analysed on a 10% SDS-PAGE gel. There was a detectable chimeric flagellin protein band for the flagellin::Sifuvirtide fusion at the size range of ˜38 kDa. The antiviral peptide FLAG×2-Sifuvirtide, was extracted and purified from B. halodurans BhFD05 culture supernatant as described in section 3.3.4. Samples were run on a 10% SDS-PAGE gel (FIG. 8).

### 4.1.3 Analysis of the N-Terminal Peptide Fusion.

The purified immunogenic chimeric flagellin fusion protein was quantified using a Qubit™ fluorometer (Invitrogen) with Quant-iT™ protein assay reagents containing a highly sensitive protein-specific fluorescent dye and certified protein standards, used according to the manufacturer's instructions. The total protein quantity obtained ranged between 1 and 5 mg immunogenic chimeric flagellin fusion protein per litre of culture supernatant, at an estimated 95% purity. The anti-viral chimeric flagellin fusion protein was electrophoresed on a 10% SDS-PAGE gel and bands were excised from the gel and subjected to enzymatic cleavage by modified sequencing grade trypsin through a method well known by those of skill in the art. The Sifuvirtide peptide presence and integrity were verified by mass-spectrometry analysis using a QSTAR® Elite mass spectrometer (Applied Biosystems/MDS SCIEX) with a discrete nano-electrospray source. Samples were loaded in Proxeon NanoES capillaries and ionized using IonSpray voltage of 900-1200 V. The instrument was calibrated using Glu-Fibrinopeptide B (Sigma-Aldrich) with fragment ions 246.15 and 1285.54 m/z. Peptide Mass Fingerprint (PMF) spectra were acquired in positive ion mode using a range of 450-1500 m/z. MS/MS data was obtained via the Information Dependent Acquisition (IDA) method where doubly and triply charged parent ions were selected for fragmentation by collision induced dissociation (CID), using nitrogen as collision gas. MS/MS data was searched against the msdb database (incorporating the sequence of flagellin, N-terminal FLAG and Sifuvirtide in the msdb.fasta file). Annotated MS/MS spectra are indicated for the identified peptides of interest with mass labels, y-ions and b-ions identified (FIGS. 9A, B).The MS/MS spectra confirmed, with extremely high confidence, the presence of two overlapping peptides (1772.8372 Da, ILEESQEQQDRNER; FIG. 9A and 2243.0567 Da, ILEESQEQQDRNERDLLE; FIG. 9B) corresponding to the C-terminal region of Sifuvirtide, providing evidence for the successful expression of Sifuvirtide as an N-terminal peptide fusion.

### Application of Host Strain: Peptides Secreted According to the Invention as C-Terminal Flagellin Fusions

### Example 5

### 5.1 Expression as FliC C-Terminal Fusion of the Anti-Viral Peptide, Sifuvirtide

5.1.1 Construction of pSECNCFSif Carrying the FLAG::Sifuvirtide Peptide.

The plasmid pSECFliC containing the full FliC protein as well as its 5′ and 3′ regions as described by Crampton et al. (2007) was used as template for the primers σ^{D}Kpn and FliCendR to obtain a 1065 by PCR product containing the full FliC protein with XhoI and BamHI sites incorporated at the C-terminal end. This fragment (digested with XhoI and KpnI) was ligated together with the FLAG-Sifuvirtide fragment (digested with XhoI and BamHI) into pSECNF2Sif digested with KpnI and BamHI. The encoded polypeptide sequence of FliC with the antiviral protein, Sifuvirtide, fused to the C-terminal end, is provided as SEQ ID NO: 14. The construct encoding this polypeptide was named pSECNCFSif and is provided as SEQ ID NO: 15. The constructs were confirmed to be correct by PCR and sequencing analysis as described for pSECNFFuzC7 and then transformed into B. halodurans BhFD05.

### 5.1.2 Evaluation of the Expression of the C-Terminal Peptide Fusion.

The chimeric flagellin gene product of pSECNCFSif was evaluated for secretion into the extracellular medium of B. halodurans BhFD05 according to section 3.3.3.

The extracellular protein fraction was analysed on a 10% SDS-PAGE gel. There was a detectable chimeric flagellin protein band for the flagellin::Sifuvirtide fusion at the size range of ˜37 kDa. The anti-viral peptide FLAG-Sifuvirtide, was extracted and purified from B. halodurans BhFD05 culture supernatant as described in section 3.3.4. Samples were run on a 10% SDS-PAGE gel (FIG. 10).

### 5.1.3 Analysis of the C-Terminal Peptide Fusion.

The purified immunogenic chimeric flagellin fusion protein was quantified using a Qubit™ fluorometer (Invitrogen) with Quant-iT™ protein assay reagents as described above. The total protein quantity obtained ranged between 5 and 12 mg immunogenic chimeric flagellin fusion protein per litre of culture supernatant, at an estimated 95% purity. The anti-viral chimeric flagellin fusion protein was electrophoresed on a 10% SDS-PAGE gel and bands were excised from the gel and subjected to enzymatic cleavage by modified sequencing grade trypsin through a method well known by those of skill in the art. The Sifuvirtide peptide presence and integrity were verified by mass-spectrometry analysis using a QSTAR® Elite mass spectrometer (Applied Biosystems/MDS SCIEX) as described in section 4.1.3. Annotated MS/MS spectra are indicated for the identified peptides of interest with mass labels, y-ions and b-ions identified (FIGS. 11A, B). The MS/MS spectra confirmed, with extremely high confidence, the presence of peptides corresponding to the N-terminal region of the anti-viral peptide LEESGADYKDDDDKGGVDMSWETWEREIENYTR (3938.6768 Da); FIG. 11A as well as the C-terminal region, ILEESQEQQDRNERDLLE (2243.0669 Da); FIG. 11B). These results provide evidence for the successful expression of Sifuvirtide as a C-terminal peptide fusion.

### REFERENCES

Bolognesi D P, Matthews T J and Wild C T (1995). Synthetic peptide inhibitors of HIV transmission. U.S. Pat. No. 5,464,933
Crampton M, Berger E, Reid S and Louw M (2007). The development of a flagellin surface display expression system in a moderate thermophile, Bacillus halodurans Alk36. Appl. Microbiol. Biotechnol. 75: 559-607.
Franquelim H G, Loura L M S, Santos N C and Castanho M A R B (2008). Sifuvirtide screens rigid membrane surfaces. Establishment of a correlation between efficacy and membrane domain selectivity among HIV fusion inhibitor peptides J. Am. Chem. Soc. 130: 6215-6223
Hancock R E W and Leherer R (1998). Cationic peptides: a new source of antibiotics. TIBTECH 16: 82-88
Hewer R and Meyer D (2003). Peptide immunogens based on the envelope region of HIV-1 are recognized by HIV/AIDS patient polyclonal antibodies and induce strong humoral immune responses in mice and rabbits. Mol Immun 40: 327-335
Selsted M E, Novotny M J, Morris W L, Tang Y-Q, Smith W and Cullor J S (1992). Indolicidin, a novel bacteriocidal tridecapeptide amide from neutrophils. J Biol Chem 267, 4292-4295
Shevchenko, A and Shevchenko, A (2001) Evaluation of the efficiency of in-gel digestion of proteins by peptide isotopic labeling and MALDI mass spectrometry, Anal Biochem 296: 279-283
Tang H-Y and Speicher D W (2004). Identification of alternative products and optimization of 2-nitro-5-thiocyanatobenzoic acid cyanylation and cleavage at cysteine residues. Anal Biochem 334: 48-61.

1. A method of producing a flagellin-based chimeric protein, the method including
culturing a B. halodurans BhFD05 (Δhag, ΔfliD, ΔwprA, Δalp, Δapr, Δvpr, Δasp) strain deposited under Accession Number 41533 at the NCIMB, and
causing the strain to express and secrete high levels of a flagellin-based chimeric protein into an extracellular growth medium,
wherein the flagellin-based chimeric protein comprises a heterologous peptide (i) inserted in-frame into a flagellin variable region which is flanked on its N-terminal side by an N-terminal fragment of a flagellin polypeptide and, optionally, flanked on its C-terminal side by a C-terminal fragment of a flagellin polypeptide, or (ii) fused to the C-terminal of a flagellin polypeptide.

2. A method according to claim 1, wherein the growth medium containing the chimeric protein, is usable as a crude preparation, with the chimeric protein being partially or fully purified from the growth medium.

3. A flagellin-based chimeric protein produced by the method of claim 1 or claim 2, and which comprises a heterologous peptide (i) inserted in-frame into a flagellin variable region which is flanked on its N-terminal side by an N-terminal fragment of a flagellin polypeptide and, optionally, flanked on its C-terminal side by a C-terminal fragment of a flagellin polypeptide, or (ii) fused to the C-terminal of a flagellin polypeptide.

4. A flagellin-based chimeric protein according to claim 3, wherein the heterologous peptide is fused only to the N-terminal fragment of a flagellin polypeptide.

5. A flagellin-based chimeric protein according to claim 3, wherein the heterologous peptide is fused to the C-terminal of a flagellin polypeptide.

6. A flagellin-based chimeric protein according to any one of claims 3 to 5 inclusive, which includes a polypeptide tag fused to the N-terminal side of the heterologous peptide.

7. A flagellin-based chimeric protein according to any one of claims 3 to 6 inclusive, which includes at least one cleavage site linked to the heterologous peptide region.

8. A flagellin-based chimeric protein according to any one of claims 3 to 7 inclusive, wherein the heterologous polypeptide is a therapeutic polypeptide.

9. A flagellin-based chimeric protein according to claim 8, wherein the heterologous peptide is an antimicrobial peptide, and is Indolicidin.

10. A flagellin-based chimeric protein according to claim 8, wherein the heterologous peptide is an antiretroviral peptide, and is Enfuvirtide.

11. A flagellin-based chimeric protein according to claim 8, wherein the heterologous peptide is an antiretroviral peptide, and is Sifuvirtide.

12. A flagellin-based chimeric protein according to claim 8, wherein the heterologous peptide is an immunogenic peptide, and is an HIV antigenic peptide.

13. Use of a therapeutic peptide according to any one of claims 3 to 12 inclusive, in the manufacture of a medicament for therapeutic use.

14. A nucleic acid encoding a chimeric protein according to any one of claims 3 to 12 inclusive, the nucleic acid comprising a nucleotide sequence encoding (i) the N-terminal fragment of a flagellin polypeptide; the variable region of a flagellin polypeptide; optionally, the C-terminal fragment of a flagellin polypeptide, and a nucleotide sequence encoding a heterologous polypeptide or therapeutic peptide inserted in-frame into the nucleotide sequence encoding the variable region of the flagellin polypeptide, or (ii) a heterologous polypeptide or therapeutic peptide fused to the C-terminal of a flagellin polypeptide.

15. A nucleic acid according to claim 14, wherein the nucleotide sequence encoding the heterologous peptide or therapeutic peptide is inserted immediately after any nucleotide between nucleotide 162 and nucleotide 606 of SEQ ID NO: 3.

16. An expression cassette which includes a nucleic acid sequence encoding the chimeric protein according to any one of claims 3 to 12 inclusive.

17. An expression cassette according to claim 16, wherein the nucleic acid sequence encoding the chimeric protein is integrated into the chromosome of the host cell.

18. A nucleic acid vector which includes a nucleic acid sequence encoding the chimeric protein of any one of claims 3 to 12 inclusive, operably linked to a transcriptional regulatory element.

19. A nucleic acid vector according to claim 18, which is an extra-chromosomal plasmid.

20. A bacterial cell containing the nucleic acid vector of claim 18 or claim 19.

21. A bacterial cell according to claim 20, which is of the strain B. halodurans Alk36 (Δhag, ΔfliD, ΔwprA, Δalp, Δapr, Δvpr, Δasp) designated BhFD05 deposited under Accession Number 41533 at the NCIMB.
